(19) United States
(12) Patent Application Publication
(10) Pub. No.: US 2018/0318129 A1
(43) Pub. Date: Nov. 8, 2018

(54) TEMPERATURE CONTROLLED THERAPEUTIC PLUSH TOY ASSEMBLY

(71) Applicant: COOL CARATS, LLC, PHOENIX, AZ (US)

(72) Inventor: JULIE KANG, PHOENIX, AZ (US)

(21) Appl. No.: 15/662,960

(22) Filed: Jul. 28, 2017

Related U.S. Application Data
(60) Provisional application No. 62/502,473, filed on May 5, 2017.

(51) Int. Cl. A61F 7/02; A63H 3/00

(52) U.S. Cl. CPC A61F 7/02; A63H 3/003; A61F 2007/0244; A61F 2007/026; A61F 2007/0201

(57) ABSTRACT

A temperature controlled therapeutic plush toy assembly is disclosed. The plush toy has a body, a thermal block contained within the body, and temperature retaining materials contained within the body. The thermal block can hold either hot or cold temperatures. The temperature of the thermal block can be modified by either placing the plush toy in the microwave or placing the plush toy in the freezer. The temperature retaining materials provide insulation to the thermal block and disperse the modified temperature of the thermal block throughout the body of the plush toy. The plush toy also has interchangeable temperature controlling garments. A heating garment is capable to heating the temperature of the body of the plush toy and a cooling garment is capable of cooling the temperature of the body of the plush toy.

### CROSS-REFERENCE TO RELATED APPLICATION

The present application claims the benefit of U.S. Provisional Application No. 62/502,473 entitled TEMPERATURE CONTROLLED THERAPEUTIC PLUSH TOY, which was filed on May 5, 2017 in the name of the inventor herein and which is incorporated in full herein by reference.

### TECHNICAL FIELD

The disclosure relates to plush toys and, more particularly to a temperature controlled therapeutic plush toy assembly.

### BACKGROUND OF THE INVENTION

People often struggle to find ways to keep themselves and especially their children cool during the hotter months of the year or, conversely, warm during the colder months. For the hotter months, some plush toys may have gel packs that are frozen separately and then inserted/removed from a pocket within the body of the plush toy. These gel packs are surrounded by stuffing and/or other materials within the body of the plush toy that neither efficiently insulate the cold gel packs nor effectively transfer the cold from the gel packs to the surface of the plush toy. Therefore, the gel packs do not stay cold for very long and they do not provide much relief to a child in hot temperatures. Furthermore, the gel packs become hard when frozen; this hardness within the body of the plush toy compromises the comfort and softness that is desired from a plush toy.

Similarly, for the colder months, some plush toys may have heating pads that are microwaved or otherwise heated separately and then inserted (and subsequently removed) from a pocket that is either located on the clothing of the plush toy or located within the body of the plush toy. For the heating pads that are inserted into the body of the plush toy, these heating pads are also surrounded by stuffing and/or other materials within the body of the plush toy that neither efficiently insulate the heating pads nor effectively transfer the heat from the heating pads to the surface of the plush toy. For the heating pads that are inserted into a pocket of the clothing of the plush toy, the clothing material is not thick enough to protect the child from experiencing burns from an overly hot heating pad.

Therefore a need exists for a temperature controlled therapeutic plush toy assembly that may provide both prolonged cooling relief to a child during the hot weather months and also provide prolonged warming relief to a child during the cold weather months. The temperature controlled therapeutic plush toy assembly also efficiently transfers cold and heat throughout its body while maintaining its softness.

### SUMMARY OF THE INVENTION

This summary is provided to introduce a selection of concepts of the present invention in a simplified form that are further described in detail below in the DETAILED DESCRIPTION OF THE INVENTION. This summary is not intended to identify each and every key feature of the invention, which remains the exclusive purview of claims, nor is the Summary intended to be used as an aid in determining the scope of the claimed subject matter.

In accordance with one embodiment of the present invention, a plush toy assembly for providing warming and cooling effects for a user is disclosed. The plush toy assembly comprises a plush toy comprising: a body; at least one thermal block contained within the body, the thermal block being capable of holding a cold temperature and also capable of holding a hot temperature; temperature retaining materials contained within the body, wherein the temperature retaining materials surround the thermal block and fill the body; wherein the temperature retaining materials insulate the thermal block and maintain a modified temperature of the thermal block while also dispersing the modified temperature of the thermal block throughout the body.

In accordance with another embodiment of the present invention, a temperature controlled therapeutic plush toy assembly is disclosed. The temperature controlled therapeutic plush toy comprises: a plush toy, the plush toy comprising: a body; at least two appendages coupled to and extending from the body; at least one thermal block contained within the body, the thermal block being capable of holding a cold temperature and also capable of holding a hot temperature; temperature retaining materials contained within the body, wherein the temperature retaining materials surround the thermal block and fill the body, and wherein the temperature retaining materials insulate the thermal block and maintain a modified temperature of the thermal block while also dispersing the modified temperature of the thermal block throughout the body; and at least one temperature controlling garment adapted to be removably worn about the body and having at least two holes formed therein so that the at least two appendages may be inserted therethrough, wherein the at least one temperature controlling garment is capable of modifying a temperature of the body.

In accordance with another embodiment of the present invention, a temperature controlled therapeutic plush toy assembly is disclosed. The temperature controlled therapeutic plush toy comprises: a plush toy, the plush toy comprising: a body; at least two appendages coupled to and extending from the body; at least one thermal block contained within the body, the thermal block being capable of holding a cold temperature and also capable of holding a hot temperature; temperature beads contained within the body, wherein the temperature beads surround the thermal block and fill the body; wherein the temperature beads insulate the thermal block and maintain a modified temperature of the thermal block while also dispersing the modified temperature of the thermal block throughout the body; and at least one temperature controlling garment adapted to be removably worn about the body and having at least two holes formed therein so that the at least two appendages may be inserted therethrough, wherein the at least one temperature controlling garment is one of a heating garment and a cooling garment, wherein the heating garment is capable of heating a temperature of the body and comprises: an outer fabric layer; a middle foam layer; and an innermost layer comprising one of a reheatable filling material and a reheatable gel pack, and wherein the cooling garment is capable of cooling the temperature of the body and comprises: an outer fabric layer; an outer middle foam layer; an inner middle foil layer comprised of thermally insulating material; and an innermost layer comprising a refreezable gel pack.

### BRIEF DESCRIPTION OF THE DRAWINGS

The novel features believed to be characteristic of the application are set forth in the appended claims. In the descriptions that follow, like parts are marked throughout the specification and drawings with the same numerals, respectively. The drawing figures are not necessarily drawn to scale and certain figures can be shown in exaggerated or generalized form in the interest of clarity and conciseness. The application itself, however, as well as a preferred mode of use, further objectives, and advantages thereof, will be best understood by reference to the following detailed description of illustrative embodiments when read in conjunction with the accompanying drawings, wherein:

FIG. 1 is a perspective view of a temperature controlled therapeutic plush toy assembly in accordance with one or more embodiments of the present invention, shown wearing a heating garment;

FIG. 2 is a partially cut-away perspective view of the plush toy of the temperature controlled therapeutic plush toy assembly of FIG. 1, shown without a garment and shown with a thermal block that is positioned within the body of the plush toy and shown with thermal beads surrounding the thermal block;

FIG. 3 is a partially cut-away perspective view of the plush toy of the temperature controlled therapeutic plush toy assembly of FIG. 1, shown without a garment and shown with multiple thermal blocks that are positioned within the body of the plush toy and shown with thermal beads surrounding and separating the thermal blocks;

FIG. 4 is a side view of the plush toy of FIG. 2, shown with a pocket formed on a portion of the body of the plush toy;

FIG. 5 cross-sectional top view of the plush toy of FIG. 4, shown with the pocket extending into the interior of the body of the plush toy and holding an absorbent material therein;

FIG. 6 is a front view of a heating garment of the temperature controlled therapeutic plush toy assembly of FIG. 1 shown with an enlarged detail view of the internal layers of the heating garment;

FIG. 7 is a front view of another embodiment of a heating garment of the temperature controlled therapeutic plush toy assembly of FIG. 1 shown with an enlarged detail view of the internal layers of the heating garment; and

FIG. 8 is a front view of a cooling garment of the temperature controlled therapeutic plush toy assembly of FIG. 1 shown with an enlarged detail view of the internal layers of the cooling garment.

### DESCRIPTION OF THE DISCLOSURE

The description set forth below is intended as a description of presently preferred embodiments of the disclosure and is not intended to represent the only forms in which the present disclosure can be constructed and/or utilized. The description sets forth the functions and the sequence of steps for constructing and operating the disclosure. It is to be understood, however, that the same or equivalent functions and sequences can be accomplished by different embodiments that are also intended to be encompassed within the spirit and scope of this disclosure.

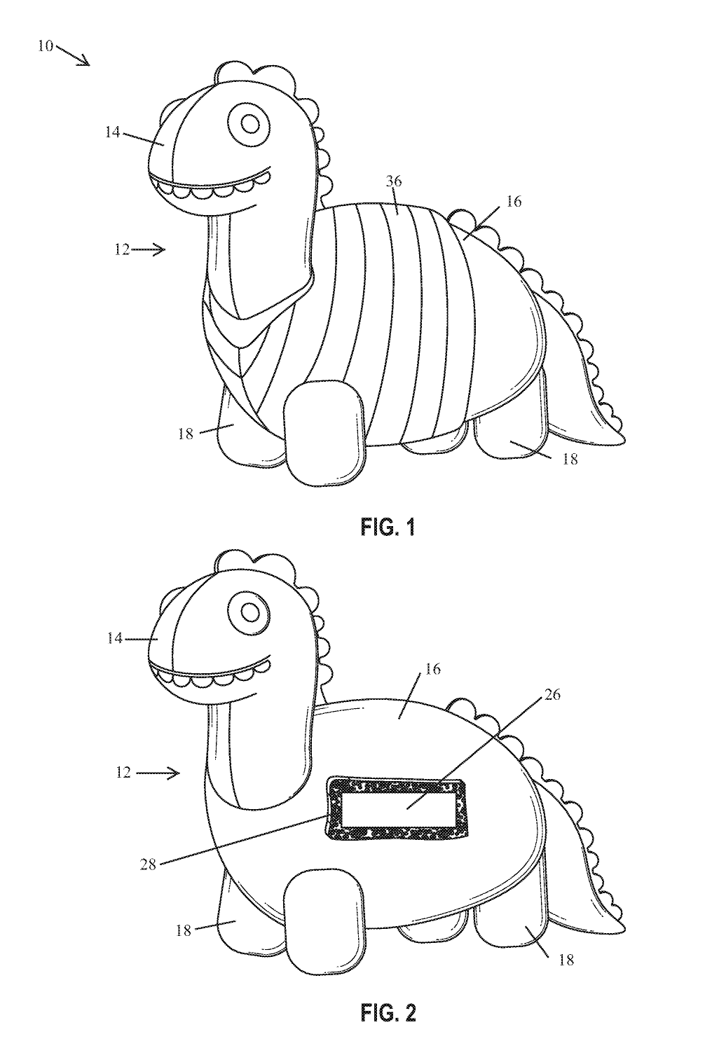

FIGS. 1-8 disclose a temperature controlled therapeutic plush toy assembly 10 of the present invention. The temperature controlled therapeutic plush toy assembly 10, in its simplest form, may comprise a plush toy 12 and at least one temperature controlling garment 36 that is adapted to be removably worn by the plush toy 12.

FIG. 1 shows one embodiment of the temperature controlled therapeutic plush toy assembly 10 wherein the plush toy 12 is wearing a temperature controlling garment 36. The plush toy 12 is shown to be in the form of a dinosaur, however, it should be clearly understood that the plush toy 12 may have the shape of any animal or of any character or object that is not an animal (e.g. an animated object, fictional character, sports ball, puppet, person, etc.). The plush toy 12 may have a head 14, a body 16, and at least two appendages 18 (i.e. arms, legs, or tail). The temperature controlling garment 36 may be worn about the body 16 of the plush toy 12 and partially held in place by the appendages 18 of the plush toy 12.

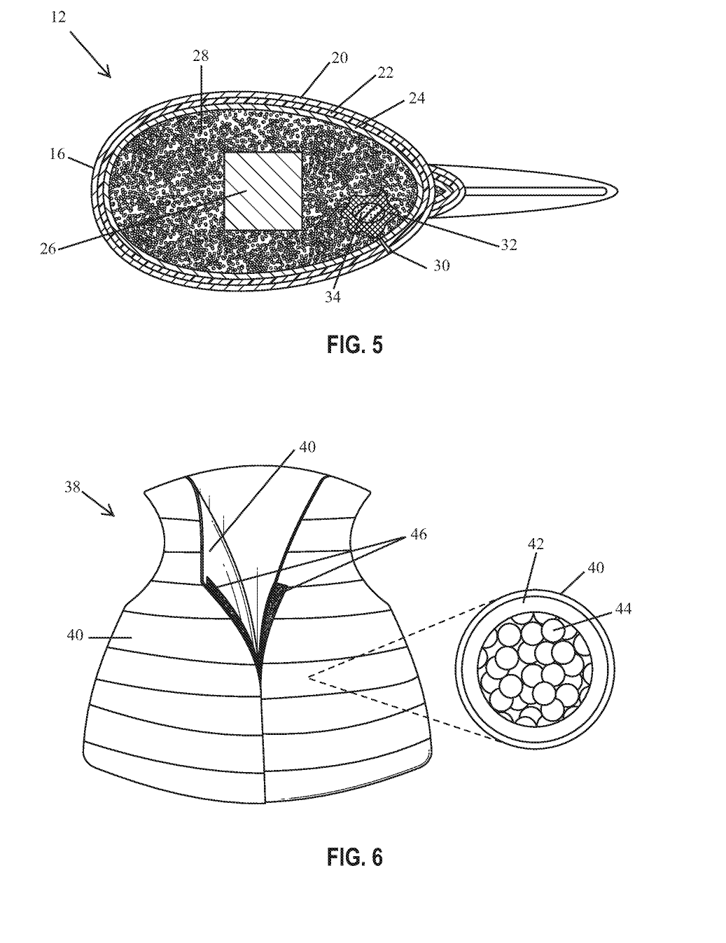

The plush toy 12 may have an outer fabric layer 20 and one or more insulation layers (see FIG. 5). The insulation layers may be present in at least the body 16 of the plush toy 12. However, it should be clearly understood that substantial benefit may also be derived if the entire plush toy 12 had insulation layers. The outer fabric layer 20 of most, or all, of the plush toy 12 may be made of cotton, flannels, soft knits, or any other suitable soft fabrics and that are not abrasive to the skin. Beneath the outer fabric layer 20 may be a middle foam layer 22 and beneath the middle foam layer 22 may be an inner foil layer 24. The inner foil layer 24 may be made of thermally insulating material such as, but not limited to, THERMAFLECT insulating fabric. The insulation layers; i.e. the middle foam layer 22 and the inner foil layer 24, help to retain the temperature of the plush toy 12, thereby prolonging the plush toy's 12 warm or cold temperatures.

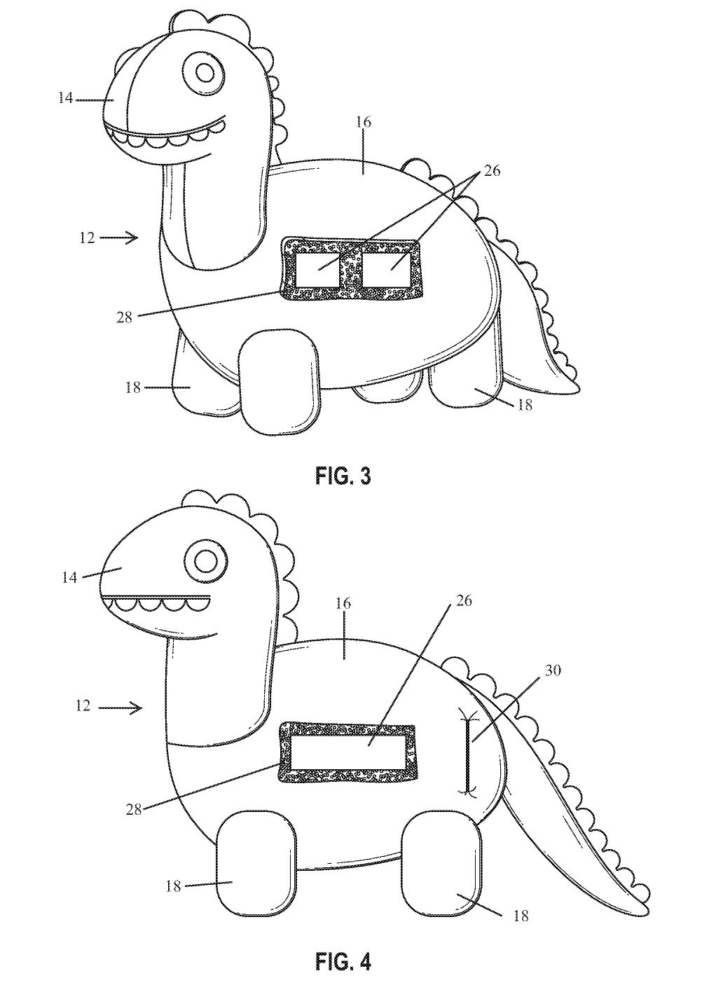

Referring to FIGS. 2-5, within the body 16 of the plush toy 12, there may be one or more thermal blocks 26. For example, FIG. 2 shows a plush toy 12 having one thermal block 26 and FIG. 3 shows a plush toy 12 having two thermal blocks 26 contained within the body 16. Substantial benefit may also be derived from more than two thermal blocks 26 being used. The thermal block 26 may hold a modified temperature that is either cold or hot. The modified temperature is either hotter or colder than the original temperature of the thermal block 26, wherein the original temperature may be equal to room temperature or ambient temperature. To achieve a modified temperature that is colder, the entire plush toy 12 may be placed in a household freezer so that the thermal block 26 may freeze. Alternatively, to achieve a modified temperature that is hotter, the entire plush toy 12 may be placed into a household microwave so that the thermal block 26 may be heated. The thermal block 26 may have a solid exterior which may be made of plastic or some other similar material and may be filled with a temperature retaining gel, such as silica gel. Silica gel hardens when it freezes, softens when it unfreezes, and may be conveniently refrozen. Similarly, the silica gel may be heated and reheated. The thermal block 26 may have any suitable shape and it may be shaped and/or positioned to conform to the shape of the body 16 of the plush toy 12; e.g. positioned vertically for a tall plush toy 12 like a giraffe or positioned horizontally for a long plush toy 12 like a dinosaur. The number of thermal blocks 26 used may also be determined by the shape of the body 16 of the plush toy 12; e.g. one thermal block 26 for the straight body 16 of a rabbit or multiple thermal blocks 26 for the curved body 16 of a snake. The thermal blocks 26 may be arranged along the length/height of the body 16 of the plush toy 12. Alternatively, the plush toy 12 may have multiple thermal blocks 26 positioned within the head 14 and/or appendages 18 of the body 16 of the plush toy 12 in order to provide maximum cooling for the entire plush toy 12.

Surrounding the thermal block 26 is a plurality of small temperature retaining materials. In this embodiment, the temperature retaining materials are temperature beads 28. The temperature beads 28 may be small, having a diameter of about 0.5-1.5 mm and the temperature beads 28 may hold cold or hot temperatures. It should be clearly understood that substantial benefit may be derived from the temperature beads 28 having a different size, as long as the temperature beads 28 are still small enough to provide a soft feel to the plush toy 12. The temperature beads 28 may be polystyrene beads which may help to insulate the thermal block 26 and maintain or prolong its cooling or heating effect while also dispersing the cold or heat from the thermal block 26 throughout the rest of the body 16 of the plush toy 12. Alternatively, the temperature beads 28 may be comprised of superabsorbent polymer beads. The temperature beads 28 may fill up the remaining area within the body 16 of the plush toy 12 around the thermal block 26. If multiple thermal blocks 26 are present, then the temperature beads 28 may also fill up the space between the thermal blocks 26. If the thermal block 26 shifts from the center of the body 16 of the plush toy 12 toward the perimeter of the body 16, then the hard thermal block 26 may cause the plush toy 12 to feel hard to the touch, thereby compromising its softness. The thermal block 26 may also rub or poke against the inside surface of the fabric of the body 16 of the plush toy 12 or against the seams holding the fabric of the body 16 together, which may lead to tearing of the fabric or ripping of the seams. Therefore, the body 16 of the plush toy 12 should be filled with enough temperature beads 28 to keep the thermal block 26 positioned near the center of the body 16. Although this embodiment describes the temperature retaining materials to be in the form of temperature beads 28, it should be clearly understood that the temperature retaining materials may also be other temperature retaining materials such as grains, rice, or flaxseed.

Referring to FIG. 4-5, the plush toy 12 may also have a pocket 30 and its opening may be formed on the outer fabric layer 20 of the body 16. The opening of the pocket 30 may be positioned at any desired place on the body 16 of the plush toy 12 and, alternatively, may be positioned on the head 14 or the appendages 18, or any other suitable part of the plush toy 12. The opening of the pocket 30 may be readily apparent or it may be hidden by having it blend in to the outside appearance of the body 16. For example, if the plush toy 12 were in the shape of a kangaroo, the pocket 30 could be constructed into the kangaroo's pouch. The opening of the pocket 30 may also have a securing device (not shown) such as a button, zipper, or hook and loop fasteners to secure the pocket 30 in a closed position.

As shown in FIG. 5, the pocket 30 may have a bag 32 made of mesh fabric, or some other porous or breathable material coupled thereto. The bag 32 is coupled to the opening of the pocket 30 and may extend toward the interior of the body 16 of the plush toy 12 so that the bag 32 directly contacts the temperature beads 28 within the body 16 of the plush toy 12. In another embodiment, the bag 32 may only extend into a location between the outer fabric layer 20 and the insulation layers. In the embodiment shown here, the bag 32 extends into the interior of the body 16 of the plush toy 12. Thus, the bag 32 also seals the opening of the pocket 30 and prevents the temperature beads 28 and thermal block 26 from falling out of the body 16 of the plush toy 12 through the hole created by the pocket 30. An absorbent material 34, such as a sponge, may be removably inserted into the pocket 30 and held within the bag 32 within the body 16 of the plush toy 12. The absorbent material 34 may be used to absorb/hold a fragrant liquid such as aromatherapy oils, perfumes, or other scented liquids. When the plush toy 12 is heated, the heat from the thermal block 26 and the temperature beads 28 may heat the absorbent material 34 within the bag 32 of the pocket 30, thereby enhancing the aroma of the fragrant liquid.

Referring to FIGS. 6-8, the temperature controlled therapeutic plush toy assembly 10 will also have at least one temperature controlling garment 36. The temperature controlling garment 36 may be used to provide either cooling effects or heating effects to the body 16 of the plush toy 12. The temperature controlling garment 36 is shown as being in the form of a vest, but it should be clearly understood that the temperature controlling garment 36 may be any type of garment such as a shirt, blouse, dress, pants, shorts, skirt, etc. and it may have any suitable shape, style, color, or design. Similar to traditional animal clothing, the temperature controlling garment 36 may have holes formed therein so that the head 14 and/or appendages 18 of the plush toy 12 may be inserted therethrough. Also similar to traditional animal clothing, the temperature controlling garment 36 may have a fastener 46, such as a button, zipper, hooks and loops, snaps, etc., to hold the temperature controlling garment 36 in place and prevent it from falling off of the plush toy 12. The fastener 46 may extend along only a portion of the length of the temperature controlling garment 36 (see FIG. 6) or the fastener 46 may extend along the full length of the temperature controlling garment 36 (see FIG. 8). It should also be understood that substantial benefit may derived from a temperature controlling garment 36 that does not have a fastener 46.

FIG. 6 shows one embodiment of a temperature controlling garment 36 that is in the form of a heating garment 38. The heating garment 38 may be close-fitting to the body 16 of the plush toy 12. The heating garment 38 may have an outer fabric layer 40 and one or more insulation layers. Here, the heating garment 38 is shown as having a middle foam layer 42 to help insulate the heat coming from the innermost layer of reheatable filling material 44. The filling material 44 may be any material that may be heated and reheated easily, such as beads, grains, rice, flaxseed, etc. The innermost layer of filling material 44 will preferably be coextensive with the middle foam layer 42 of the heating garment 38, rather than being confined within a certain portion or pocket of the heating garment 38. The filling material 44 is firmly and permanent held within the heating garment 38 by the middle foam layer and the outer fabric layer 40; i.e. the filling material 44 is not removable from the heating garment 38 (removable filling material 44 may prove to be a safety concern for young children using the temperature controlled therapeutic plush toy assembly 10). Therefore, in order to heat the filling material 44, the entire heating garment 38 may be placed within a microwave. Once the heating garment 38 has been heated, it may then be placed on the plush toy 12. The heat from the heating garment 38 modifies the temperature of the body 16 of the plush toy 12 by heating it.

Although the temperature controlling garment 36 is shown as being a sleeveless heating garment 38, it should be clearly understood that the temperature controlling garment 36 may have a hood, sleeves, or other coverings for the head or appendages 18 of the plush toy 12. If the temperature controlling garment 36 has a hood, sleeves, or other head or appendage coverings, the filling material 44 will preferably be only present in the area of the temperature controlling garment 36 that covers the body 16 of the plush toy 12; i.e. the filling material 44 preferably will not be present in the hood, sleeves, or other head or appendage coverings. However, it should be clearly understood that substantial benefit may nevertheless be derived if the filling material 44 is also present in the hood, sleeves, or other head or appendage coverings.

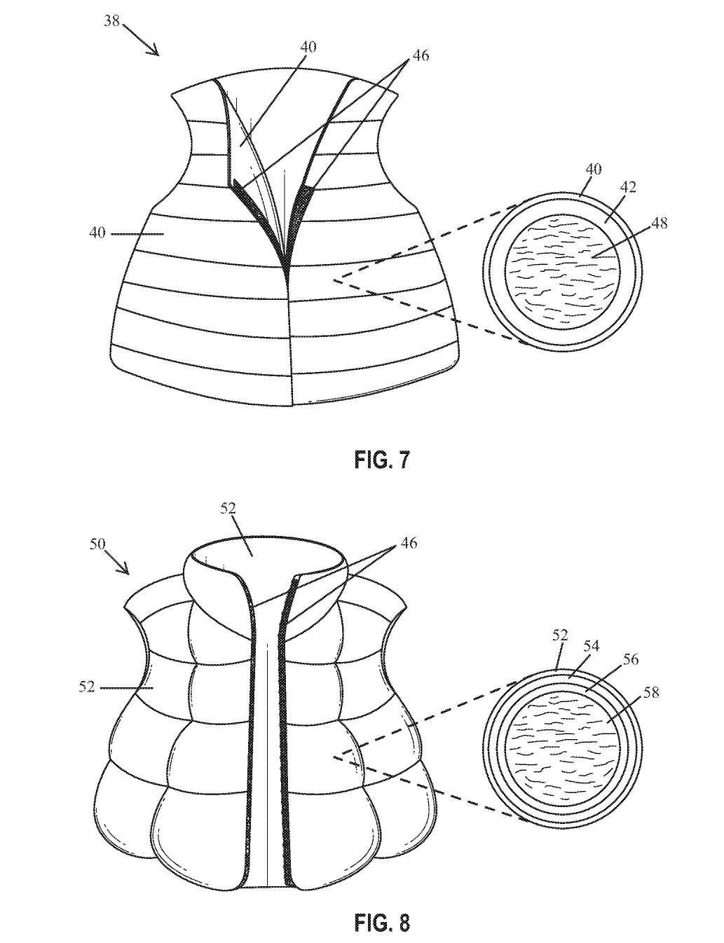

FIG. 7 shows an alternative embodiment of the heating garment 38. This embodiment is similar to that shown in FIG. 6, except that in this embodiment, the innermost layer comprises a reheatable gel pack 48 instead of filling material 44. The reheatable gel pack 48 will preferably be coextensive with the middle foam layer 42 of the heating garment 38, rather than being confined within a certain portion or pocket of the heating garment 38. Preferably, the reheatable gel pack 48 is also firmly and permanently held within the heating garment 38 by the middle foam layer and the outer fabric layer 40; i.e. the reheatable gel pack 48 is, preferably, not removable from the heating garment 38 (a removable reheatable gel pack 48 may prove to be a safety concern for young children using the temperature controlled therapeutic plush toy assembly 10). Nonetheless, it should be clearly understood that substantial benefit may still be derived if the reheatable gel pack 48 (or filling material 44) is capable of being removed from the heating garment 38.

FIG. 8 shows one embodiment of a temperature controlling garment 36 that is in the form of a cooling garment 50. The cooling garment 50 may be close-fitting to the body 16 of the plush toy 12. The cooling garment 50 may have an outer fabric layer 52 and one or more insulation layers. Here, the cooling garment 50 is shown as having an outer middle foam layer 54 to insulate the cold coming from the innermost layer which comprises a refreezable gel pack 58 and to reduce condensation. The cooling garment 50 also has an inner middle foil layer 56 which helps to insulate the cold coming from the refreezable gel pack 58 and to reflect heat. The inner middle foil layer 56 may be made of thermally insulating material such as, but not limited to, THERMAFLECT insulating fabric. The refreezable gel pack 58 will preferably be coextensive with the inner middle foil layer 56 of the cooling garment 50, rather than being confined within a certain portion or pocket of the cooling garment 50. The refreezable gel pack 58 is firmly and permanent held within the cooling garment 50 by the inner middle foil layer 56, the outer middle foam layer 54, and the outer fabric layer 52; i.e. the refreezable gel pack 58 is not removable from the cooling garment 50 (a removable refreezable gel pack 58 may prove to be a safety concern for young children using the temperature controlled therapeutic plush toy assembly 10). Therefore, in order to freeze the refreezable gel pack 58, the entire cooling garment 50 may be placed within a freezer. Once the cooling garment 50 has been cooled, it may then be placed on the plush toy 12. The cold from the cooling garment 50 modifies the temperature of the body 16 of the plush toy 12 by cooling it. Although it is preferable that the refreezable gel pack 58 is not removable from the cooling garment 50, it should be clearly understood that substantial benefit may still be derived if the refreezable gel pack 58 is capable of being removed from the cooling garment 50.

Although the temperature controlling garment 36 is shown as being a sleeveless cooling garment 50, it should be clearly understood that the temperature controlling garment 36 may have a hood, sleeves, or other coverings for the head or appendages 18 of the plush toy 12. If the temperature controlling garment 36 has a hood, sleeves, or other head or appendage coverings, the refreezable gel pack 58 will preferably be only present in the area of the temperature controlling garment 36 that covers the body 16 of the plush toy 12; i.e. the refreezable gel pack 58 preferably will not be present in the hood, sleeves, or other head or appendage coverings. However, it should be clearly understood that substantial benefit may nevertheless be obtained from the refreezable gel pack 58 also being present in the hood, sleeves, or other head or appendage coverings.

### Statement of Operation

Desert climate areas, like Arizona, can experience extreme heat during the summer months. Unexpectedly, these desert climate areas may also experience cold winter months. People often struggle to find ways to keep themselves and especially their children cool during the hotter months and, conversely, warm during the colder months. Rather than having to use two separate plush toys, the temperature controlled plush toy assembly 10 of the present invention may be used for both purposes; providing either cooling effects or warming effects for the comfort of the user. The plush toy 12 may be heated by placing the plush toy 12 (with or without the heating garment 38) into the microwave or the plush toy 12 may be cooled by placing the plush toy 12 (with or without the cooling garment 50) into the freezer. If a user does not wish to place the plush toy 12 in the microwave, the heating garment 38 alone may be placed into the microwave and then, when hot, placed about the body 16 of the plush toy 12 to heat the plush toy 12. And if the user does not wish to place the plush toy 12 in the freezer, the cooling garment 50 alone may be placed into the freezer and then, when cold, placed about the body 16 of the plush toy 12 to cool the plush toy 12. The heating garment 38 and cooling garment 50 are configured to be interchangeable for the plush toy 12, depending upon whether the plush toy 12 is to have a heating effect or a cooling affect for the user.

The temperature controlling garment 36 affects the temperature of the body 16 of the plush toy 12. When the temperature controlling garment 36 is placed on the plush toy 12, the temperature of the plush toy 12 adjusts to the temperature of the temperature controlling garment 36. The outward appearance of the temperature controlling garment 36 may be designed to indicate whether the temperature controlling garment 36 is a heating garment 38 or a cooling garment 50. For example, the heating garment 38 may use a thin outer fabric layer 40 to convey that the heating garment 38 is a type of thin garment that one might wear during hot weather. The notion of hot weather may remind the user that the heating garment 38 is the one that should be placed in the microwave. Conversely, the cooling garment 50 may use a thick outer fabric layer 52 to convey that the cooling garment 50 is a type of thick garment that one might wear during the cold weather. The notion of cold weather may remind the user that the cooling garment 50 is the one that should be placed in the freezer.

During the hotter months, the user may place the entire cooling garment 50 into a freezer so that the refreezable gel pack 58 contained within the cooling garment 50 will become frozen. Once the cooling garment 50 is placed on the plush toy 12, the temperature of the plush 12 toy will become cool and the temperature beads 28 and thermal block 26 inside of the plush toy 12 will further retain the temperature of the cooling garment 50.

If the user wishes to enhance the cooling effect of the temperature controlled therapeutic plush toy assembly 10 (perhaps in cold climate areas that experience extremely cold temperatures), the user may place the entire plush toy 12 (with or without the cooling garment 50) into the freezer so that the temperature block 26 will become frozen. For maximum and prolonged cooling effects, the user may place the plush toy 12 and the cooling garment 50 into the freezer (either coupled together or positioned separately within the freezer). The user may then place the cooling garment 50 onto the plush toy 12 prior to use.

During the colder months, the user may place the entire heating garment 38 into a microwave so that the filling material 44 or the reheatable gel pack 48 contained within the heating garment 38 will become hot. Once the heating garment 38 is placed on the plush toy 12, the temperature of the plush 12 toy will become warm and the temperature beads 28 and thermal block 26 inside of the plush toy 12 will further retain the temperature of the heating garment 38.

If the user wishes to enhance the heating effect of the temperature controlled therapeutic plush toy assembly 10 (perhaps in desert climate areas that experience extremely hot temperatures), the user may place the entire plush toy 12 (with or without the heating garment 38) into the microwave so that the temperature block 26 may be heated. For maximum and prolonged heating effects, the user may place the plush toy 12 and the heating garment 38 into the microwave (either coupled together or positioned separately within the microwave). The user may then place the heating garment 38 onto the plush toy 12 prior to use.

A user may also wish to use essential oils, aromatherapy oils, or other scented liquids with the temperature controlled therapeutic plush toy assembly 10. To do so, the user may open the pocket 30 on the outer fabric layer 20 of the body 16 of the plush toy 12. The user may then apply the oils or scented liquids to the absorbent material 34 and place the absorbent material 34 back into the bag 32 of the pocket 30, and close the pocket 30. Alternatively, the user may place a vial of the oils or scented liquids directly into the pocket 30 without having to use the absorbent material 34.

The foregoing description is illustrative of particular embodiments of the application, but is not meant to be limitation upon the practice thereof. While the invention has been illustrated with respect to several specific embodiments thereof, these embodiments should be considered as illustrative rather than limiting. Various modifications and additions may be made and will be apparent to those skilled in the art. Accordingly, the invention should not be limited by the foregoing description, but rather should be defined only by the following claims.

What is claimed is:

1. A plush toy assembly for providing warming and cooling effects for a user comprising:
a plush toy, the plush toy comprising:
a body;
at least one thermal block contained within the body, the thermal block being capable of holding a cold temperature and also capable of holding a hot temperature;
temperature retaining materials contained within the body, wherein the temperature retaining materials surround the thermal block and fill the body,
wherein the temperature retaining materials insulate the thermal block and maintain a modified temperature of the thermal block while also dispersing the modified temperature of the thermal block throughout the body.

2. The plush toy assembly of claim 1, wherein the body of the plush toy further comprises:
an outer fabric layer;
an inner foil layer; and
a middle foam insulation layer positioned between the outer fabric layer and the inner foil layer.

3. The plush toy assembly of claim 2, wherein the body of the plush toy further comprises a pocket, the pocket comprising:
an opening formed on the outer fabric layer of the body;
a bag coupled to the opening and extending into the body of the plush toy; and
a removable absorbent material contained within the bag, wherein the absorbent material contains fragrant liquid.

4. The plush toy assembly of claim 1, wherein the thermal block comprises a solid exterior that is filled with a temperature retaining gel that is capable of holding hot and cold temperatures.

5. The plush toy assembly of claim 1, wherein the temperature retaining materials comprise temperature beads, wherein the temperature beads are capable of holding hot and cold temperatures.

6. The plush toy assembly of claim 1, further comprising:
at least two appendages coupled to and extending from the body; and
at least one temperature controlling garment adapted to be removably worn about the body and having at least two holes formed therein so that the at least two appendages may be inserted therethrough, wherein the at least one temperature controlling garment is capable of modifying a temperature of the body.

7. The plush toy assembly of claim 6, wherein the temperature controlling garment is a heating garment, the heating garment comprising:
an outer fabric layer;
a middle foam layer; and
an innermost layer comprising one of a reheatable filling material and a reheatable gel pack,
wherein the heating garment heats the temperature of the body.

8. The plush toy assembly of claim 6, wherein the temperature controlling garment is a cooling garment, the cooling garment comprising:
an outer fabric layer;
an outer middle foam layer;
an inner middle foil layer; and
an innermost layer comprising a refreezable gel pack,
wherein the cooling garment cools the temperature of the body.

9. The plush toy assembly of claim 6, wherein an outward appearance of the temperature controlling garment indicates whether the temperature controlling garment is a heating garment or a cooling garment.

10. The plush toy assembly of claim 6, wherein the modified temperature of the thermal block is achieved by one of:
heating at least one of the plush toy and the temperature controlling garment in a microwave; and
freezing at least one of the plush toy and the temperature controlling garment in a freezer.

11. A temperature controlled therapeutic plush toy assembly comprising:
a plush toy, the plush toy comprising:
a body;
at least two appendages coupled to and extending from the body;
at least one thermal block contained within the body, the thermal block being capable of holding a cold temperature and also capable of holding a hot temperature;
temperature retaining materials contained within the body, wherein the temperature retaining materials surround the thermal block and fill the body, and wherein the temperature retaining materials insulate the thermal block and maintain a modified temperature of the thermal block while also dispersing the modified temperature of the thermal block throughout the body; and
at least one temperature controlling garment adapted to be removably worn about the body and having at least two holes formed therein so that the at least two appendages may be inserted therethrough, wherein the at least one temperature controlling garment is capable of modifying a temperature of the body.

12. The plush toy of claim 11, wherein the body of the plush toy further comprises:
an outer fabric layer;
an inner foil layer comprised of thermally insulating material; and
a middle foam insulation layer positioned between the outer fabric layer and the inner foil layer.

13. The plush toy of claim 11, wherein the thermal block is positioned to conform to a shape of the body of the plush toy and comprises a solid exterior that is filled with a temperature retaining gel that is capable of holding hot and cold temperatures.

14. The plush toy of claim 11, wherein the temperature retaining materials comprise temperature beads, wherein the temperature beads have a diameter of about 0.5-1.5 mm and are capable of holding hot and cold temperatures.

15. The plush toy of claim 11, wherein the temperature controlling garment is a heating garment, the heating garment comprising:
an outer fabric layer;
a middle foam layer; and
an innermost layer comprising one of a reheatable filling material and a reheatable gel pack,
wherein the heating garment heats the temperature of the body.

16. The plush toy of claim 11, wherein the temperature controlling garment is a cooling garment, the cooling garment comprising:
an outer fabric layer;
an outer middle foam layer;
an inner middle foil layer comprised of thermally insulating material; and
an innermost layer comprising a refreezable gel pack,
wherein the cooling garment cools the temperature of the body.

17. The plush toy of claim 11 wherein an outward appearance of the temperature controlling garment indicates whether the temperature controlling garment is a heating garment or a cooling garment.

18. The plush toy of claim 11, wherein the modified temperature of the thermal block is achieved by one of:
heating at least one of the plush toy and the temperature controlling garment in a microwave; and
freezing at least one of the plush toy and the temperature controlling garment in a freezer.

19. The plush toy of claim 11, wherein the body of the plush toy further comprises a pocket, the pocket comprising:
an opening formed on the outer fabric layer of the body;
a porous bag coupled to the opening and extending into the body of the plush toy; and
a removable sponge contained within the bag, wherein the sponge contains fragrant liquid.

20. A temperature controlled therapeutic plush toy assembly comprising:
a plush toy, the plush toy comprising:
a body;
at least two appendages coupled to and extending from the body;
at least one thermal block contained within the body, the thermal block being capable of holding a cold temperature and also capable of holding a hot temperature;
temperature beads contained within the body, wherein the temperature beads surround the thermal block and fill the body;
wherein the temperature beads insulate the thermal block and maintain a modified temperature of the thermal block while also dispersing the modified temperature of the thermal block throughout the body; and
at least one temperature controlling garment adapted to be removably worn about the body and having at least two holes formed therein so that the at least two appendages may be inserted therethrough, wherein the at least one temperature controlling garment is one of a heating garment and a cooling garment,
wherein the heating garment is capable of heating a temperature of the body and comprises:
an outer fabric layer;
a middle foam layer; and
an innermost layer comprising one of a reheatable filling material and a reheatable gel pack, and
wherein the cooling garment is capable of cooling the temperature of the body and comprises:
an outer fabric layer;
an outer middle foam layer;
an inner middle foil layer comprised of thermally insulating material; and
an innermost layer comprising a refreezable gel pack.
